(19) United States
(12) Patent Application Publication
(10) Pub. No.: US 2020/0200778 A1
(43) Pub. Date: Jun. 25, 2020

(54) OPTICAL DEVICE FOR DETECTING AND QUANTIFYING VOLATILE COMPOUNDS

(71) Applicants: UNIVERSiTE D'AIX-MARSEILLE, MARSEILLE 7 (FR); Centre National De La Recherche Scientifique, Paris (FR)

(72) Inventors: Elsa Alvarez, MARSEILLE (FR); David GROSSO, ALLAUCH (FR); Marco ABBARCHI, MARSEILLE (FR)

(21) Appl. No.: 16/615,486

(22) PCT Filed: May 15, 2018
(86) PCT No.: PCT/EP2018/062605

§ 371 Date: Nov. 21, 2019

(30) Foreign Application Priority Data
May 22, 2017 (FR) 1754533

(51) Int. Cl. G01N 33/98; G01N 21/77; G01N 33/497; G01N 31/22

(52) U.S. Cl. CPC G01N 33/98; G01N 31/223; G01N 33/4972; G01N 21/77

(57) ABSTRACT

The invention relates to an optical device for detecting and quantifying volatile compounds, comprising:
a sensitive reflective element (21), the reflection rate of which varies as a function of the ethanol content contained in an atmosphere to be tested, the sensitive reflective element (21) comprising:
   a substrate (27),
   a sensitive layer (29) comprising microporous hydrophobic sol-gel silica,
a light source (23) arranged to illuminate the sensitive layer (29) under an incident angle,
a light detector (25) for measuring the intensity reflected by the reflective element (21) under an angle of detection,
and
a processing and calculation unit (31) configured to deduce from the intensity reflected by the reflective element (11) a parameter corresponding to a blood alcohol content.

The present invention relates to an optical device for detecting and quantifying volatile compounds such as alcohol and determining alcohol in the form of the blood alcohol level.

Alcohol consumption by motorists is a major challenge for society. In 2015 in France, alcohol was responsible for close to one third of deadly accidents and consequently constitutes one of the primary causes of mortality on the roads. This number represents more than 1000 individuals killed each year. At night, excessive alcohol consumption is involved in half of deadly accidents.

For this reason, alcohol consumption is prohibited in certain states or at the least strictly limited, notably with respect to young drivers.

Numerous measuring and verifying devices, in particular breathalyzers, are known for verifying the blood alcohol content of a road user. These devices measure the ethanol content contained in the breath exhaled by an individual, thereby making it possible to determine the blood alcohol content thereof in a certain range.

Among these breathalyzers, chemical breathalyzers and electronic breathalyzers are in particular distinguished.

For chemical breathalyzers, they operate according to the principle of oxidation-reduction which takes place between potassium dichromate, sulfuric acid and ethanol. It is thus a concrete application of organic chemistry. Indeed, a green/blue coloration of the cotton wool is observed at the end of the reaction. Thus, ethanol and potassium dichromate have reacted together: the test is positive. These tests are inexpensive as such, but are single use. Furthermore, these tests have only a limited shelf life. Finally, these tests in truth give an indication of the blood alcohol content and have a low measurement accuracy.

Electronic breathalyzers make it possible to solve a certain number of these drawbacks.

The electronic breathalyzer analyses the exhaled alveolar air by virtue of a semiconductor detector produced in the form of an ethanol-sensitive electrochemical sensor. In the case of the electronic breathalyzer, a current is created when the presence of ethanol molecules in the breath is relatively high. Thus, the intensity of the current is linked to the amount of ethanol in the exhaled air. The semiconductor then allows the current generated to pass to a microprocessor which evaluates it and converts it into a measurement that can be read on a display.

Electronic breathalyzers are reliable and accurate. They can be used repeatedly with various road users, only a small plastic mouthpiece intended to be in contact with the mouth of the user is disposable and single use.

However, these electronic breathalyzers are very complex and expensive in order to achieve the required accuracy. Furthermore, their response time is quite weak, typically between 3 s and 5 s.

An object of the present invention is to provide a reusable and less expensive alternative to semiconductor electronic breathalyzers which exhibits good measurement reliability.

To this effect, the present invention provides an optical device for detecting and quantifying volatile compounds, comprising:
a sensitive reflective element, the reflection rate of which varies as a function of the ethanol content contained in an atmosphere to be tested, the sensitive reflective element comprising:
   a substrate,
   a sensitive layer comprising microporous hydrophobic sol-gel silica having a thickness of greater than 250 nm, notably of between 400 nm and 1200 nm, a mean pore size of less than 2 nm and a porosity of less than 25%, the sensitive layer being intended to be placed in the presence of an atmosphere to be tested, notably which may or may not be charged with ethanol, and more particularly the breath exhaled from an individual,
a monochromatic or quasi-monochromatic light source arranged to illuminate the sensitive layer under an incident angle,
a light detector for measuring the intensity reflected by the reflective element under an angle of detection,

and
a processing and calculation unit configured to deduce from the intensity reflected by the reflective element a parameter corresponding to a blood alcohol content.

By virtue of the invention, an optical device for detecting and quantifying volatile compounds such as alcohol, and notably a device for determining the blood alcohol content, which is efficient and very sensitive with an excellent response time for detecting the blood alcohol content of an individual, is available.

The device according to the invention can also have one or more of the following features taken alone or in combination:

It is possible for the sensitive layer not to comprise structuring agents, notably CTAB, DTAB or F127.

The substrate has, according to one example, a refractive index greater than 1.8, preferably greater than 2.5, notably greater than 3 for a wavelength of between 250 nm and 1500 nm.

The substrate is for example made of a semiconductive material, notably of silicon.

The incident angle and the angle of detection are respectively notably between 30° and 75°.

The wavelength of the light source is monochromatic and chosen with the angle of incidence so as to coincide with the position in terms of wavelength of an inflection (I₁, I₂, I₃) between two constructive and destructive interference peaks of the reflection spectrum of the reflective element.

The wavelength of the light source is between 500 nm and 1000 nm.

The sensitive layer has a refractive index of between 1.2 and 1.6, more particularly between 1.3 and 1.4 for a wavelength of between 500 nm and 1000 nm.

The detection device may also comprise
an additional sensitive reflective element, the reflection rate of which varies as a function of the moisture content contained in an atmosphere to be tested,
   an additional light detector for measuring the intensity reflected by the additional sensitive reflective element under an angle of detection,
   the processing and calculation unit being configured to deduce from the intensities reflected, on the one hand, by the reflective element and, on the other hand, by the additional sensitive reflective element a parameter corresponding to a blood alcohol content while taking into account the influence of the moisture in the atmosphere to be tested.

The invention also relates to a process for producing a sensitive reflective element for an optical device for detecting and quantifying volatile compounds, as defined above, characterized in that
a sol-gel solution is prepared by dissolving TEOS and MTEOS in a solution composed of PrOH, hydrochloric acid and water,
the sol-gel solution is deposited on the substrate under a relative humidity of between 40% and 90%, notably between 50% and 60% in order to obtain a sensitive layer,
the sensitive layer is subjected to a calcining step at a temperature of between 250° C. and 450° C., notably at 350° C., this being for a period of time greater than 5 min, notably for 10 min.
The process may have one or more of the following features taken alone or in combination:

The thickness of the sensitive layer is for example greater than 250 nm, notably between 400 nm and 1200 nm.

The invention also relates to a sensitive reflective element for an optical device for detecting and quantifying volatile compounds, as defined above, characterized in that the reflective element comprises
a substrate, and
a sensitive layer comprising microporous hydrophobic sol-gel silica having a thickness of greater than 250 nm, notably of between 400 nm and 1200 nm, a mean pore size of less than 2 nm and a porosity of less than 25%, the sensitive layer being intended to be placed in the presence of an atmosphere to be tested, notably which may or may not be charged with ethanol, and more particularly the breath exhaled from an individual.

The sensitive reflective element has a reflection rate which varies as a function of the ethanol content contained in an atmosphere to be tested.

The invention also relates to a process for producing an optical detection device for detecting and quantifying volatile compounds, as defined above, characterized in that
a sensitive layer is deposited on a substrate in order to form a sensitive reflective element,
the reflection spectrum of the sensitive reflective element is determined,
the wavelength of the light source is monochromatic, or quasi-monochromatic, and chosen with the angle of incidence of the light ray on the reflective element so as to coincide with the position in terms of wavelength of an inflection of the reflection spectrum of the reflective element.

According to one aspect of this process, the substrate has for example a refractive index of between 1.8 and 4, notably between 2.5 and 3.5, the sensitive layer has a thickness of greater than 250 nm, notably of between 400 nm and 1200 nm, and has a refractive index of between 1.2 and 1.6, more particularly between 1.3 and 1.4 and the wavelength of the light source is between 500 nm and 1000 nm.

According to another aspect, the reflective element is produced according to the process as defined above.

The invention also relates to an optical detection process for detecting and quantifying volatile compounds in an atmosphere to be tested, characterized in that it comprises the following steps:
the intensity reflected by a sensitive reflective element as defined above, the reflection rate of which varies as a function of the ethanol content contained in the atmosphere to be tested, is measured,
the intensity reflected by an additional sensitive reflective element as defined above, the reflection rate of which varies as a function of the moisture content contained in the atmosphere to be tested, is measured,
in a predetermined time zone of interest for which the time derivative is the same for the two intensities measured in the case of the absence of ethanol, the difference in the time derivative of the two intensities measured is determined in order to carry out a correlation with a calibration curve in order to quantify the ethanol contained in the atmosphere to be tested.

The wavelength of the light source is chosen so as to coincide with the position in terms of wavelength of an inflection between two constructive and destructive interference peaks of the reflection spectrum of the sensitive reflective element.

Other advantages and characteristics will emerge on reading the description of the invention, and also the following figures in which:

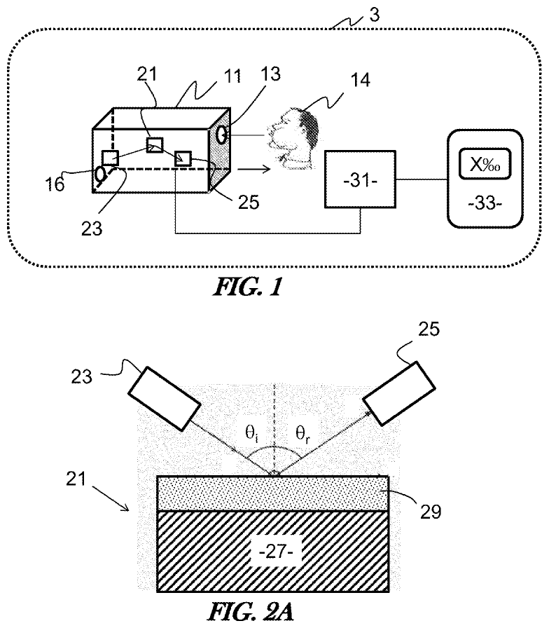

FIG. 1 shows a simplified diagram of a device for detecting and quantifying volatile compounds according to the invention, notably for determining a blood alcohol content,

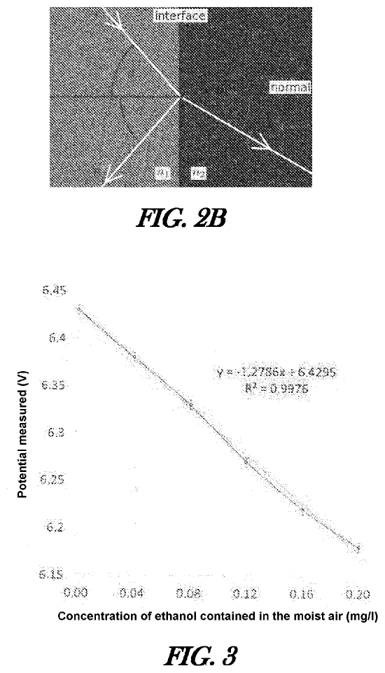

FIG. 2A shows an enlarged schematic diagram of the detection portion of the device of FIG. 1,

FIG. 2B shows a schematic diagram to illustrate the Fresnel equations,

FIG. 3 represents a graph showing a measurement parameter representing the intensity of the reflected light signal as a function of the ethanol concentration in the moist air,

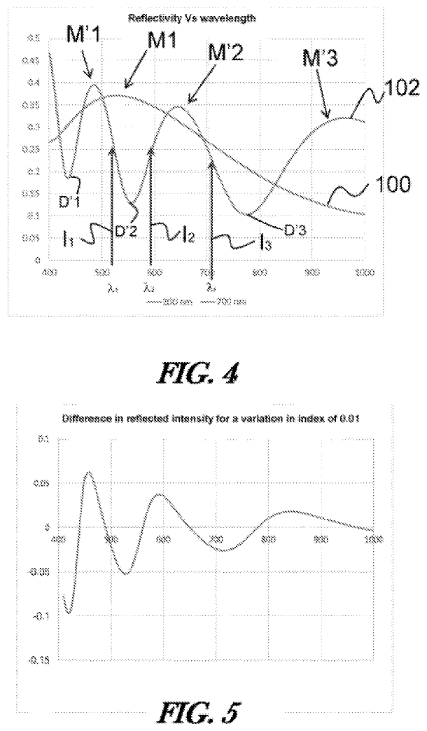

FIG. 4 represents a graph showing, for two different thicknesses, the reflectivity as a function of wavelength, and

FIG. 5 shows, on a graph, the variation in the reflected intensity as a function of the wavelength for a layer of a given thickness when the latter undergoes a variation in refractive index by a predefined value,

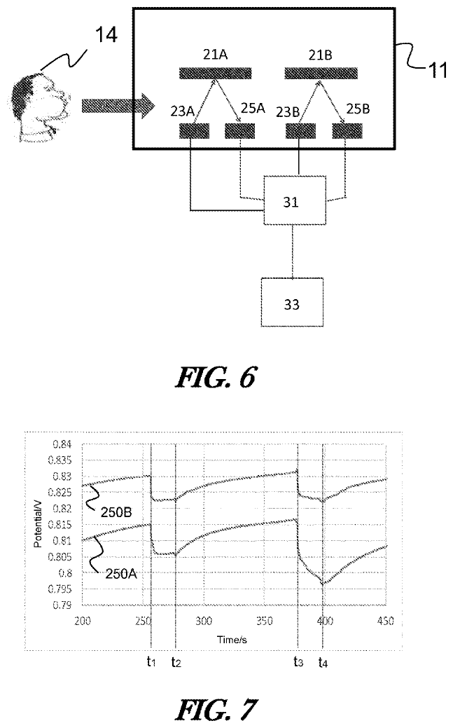

FIG. 6 shows, schematically, another embodiment of the device for detecting and quantifying volatile compounds according to the invention, notably for determining a blood alcohol content,

FIG. 7 shows, on a graph, the measurement parameters as a function of time,

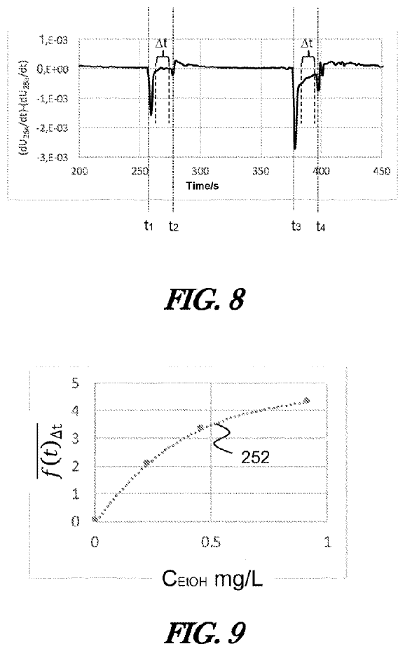

FIG. 8 shows, on a graph as a function of time, a processing of the measurement parameters of FIG. 7,

FIG. 9 shows a correlation curve between, on the one hand, an ethanol content and, on the other hand, measurement values processed according to FIG. 8.

In all the figures, the identical elements bear the same reference numbers.

The following implementations are examples. Although the description refers to one or more embodiments, this does not necessarily mean that each reference relates to the same embodiment, or that the characteristics apply only to a single embodiment. Simple characteristics of the various embodiments can also be combined and/or interchanged so as to provide other implementations.

FIG. 1 shows schematically a side view of an optical detection device 3 for detecting and quantifying volatile compounds, notably with a view to determining a blood alcohol content.

This optical device 3 comprises a measuring chamber 11 with an inlet 13 intended to receive a flow of atmosphere to be tested, notably an alveolar flow from an individual 14 exhaling their breath.

This measuring chamber 11 can for example be made of plastic and its dimensions are, for example, chosen so as to contain a slightly smaller volume than the average volume expired by an individual 14, so that, during a blood alcohol content check, the individual 14 blows into the chamber 11 and the air contained in the chamber 11 is completely replaced with the alveolar air from the exhaling individual 14.

For hygiene reasons, the inlet of a single-use mouthpiece (not represented) which attaches, for example, by snap-fastening on the inlet 13 of the chamber 11 is provided for.

The back wall of the chamber 11 opposite that which has the inlet 13 comprises an outlet 16 for the alveolar air.

In FIG. 1, the chamber 11 has a simple parallelepipedal shape, but other shapes can be envisioned, notably in order to be able to thoroughly mix the flow entering via the inlet 13. Thus, the chamber 11 can comprise deflectors (not represented) for creating a swirling flow to thoroughly mix the entering flow. The same effect can be obtained for example by a particular shape of the walls of the chamber 11, for example in the shape of a volute.

According to one development, at the outlet 16, it is possible to install an air blower, for example moved by an electric motor, in order to be able to purge the air in the chamber 11. This is because, after a measurement and in order to make the device 3 operational as rapidly as possible once again, it is necessary to replace the atmosphere for example with ethanol-free ambient air.

In the present case, the walls of the measuring chamber 11 are for example made of an opaque material, which is notably black, for example of polycarbonate or poly(methyl methacrylate) charged with carbon black so as to prevent ambient from being able to penetrate into the chamber 11 in order to reduce the risk of disruption of the measurements by ambient light.

It is moreover possible to provide, for the internal walls of the chamber 11, a light-absorbing coating to further reduce the risk of disruption of the measurements by ambient light.

Arranged inside the chamber 11 are a sensitive reflective element 21, the reflection rate of which varies as a function of the ethanol content contained in an atmosphere to be tested, a monochromatic light source 23 arranged to illuminate the sensitive reflective element 21 under an incident angle and a light detector 25 for measuring the intensity reflected by the sensitive reflective element 21.

For example, the monochromatic light source 23 and the light detector 25 are on one wall of the chamber 11, while the sensitive reflective element 21 is on the opposite wall of the chamber 11.

Of course, other assemblies are possible provided that the light detector 25 can detect the light reflected by the reflective element 21.

The monochromatic light source 23 is for example a laser, in particular a laser diode or an LED. The wavelength is for example between 250 nm and 1200 nm approximately. The light source 23 can emit a monochromatic flux of light continuously or in a pulsed manner.

The light detector 25 may be any image-taking apparatus capable of measuring the intensity reflected by the sensitive reflective element 21, such as for example a camera. According to one preferential embodiment, the light detector 25 is a silicon photodiode.

The sensitive reflective element 21 has a reflection rate which varies proportionally as a function of the ethanol content contained in an atmosphere to be tested in the chamber 11 in order to be able to measure the ethanol content of the atmosphere made up of the alveolar flow from the individual 14 exhaling their breath so as to be able to determine the blood alcohol content of said person by virtue of the detection of the light intensity reflected by this reflective element 21.

To this effect, as shown in greater detail in FIG. 2, the sensitive reflective element 21 comprises a substrate 27 and a sensitive layer 29.

The substrate 27 has a refractive index, in particular a refractive index greater than 1.8, preferably greater than 2.5, notably greater than 3.4 for a wavelength of between 400 nm and 1500 nm.

According to one embodiment, the substrate 27 is made of a semiconductive material, notably of silicon. As an alternative, the substrate 27 may also be made of glass, the back face of which, that is to say the face opposite the light source 23 and the light detector 25, is reflective, for example because of a metal coating notably of the mirror type.

Deposited on this substrate 27 is a sensitive layer 29 of microporous sol-gel silica having a thickness of greater than 250 nm, notably of between 400 nm and 1200 nm, a mean pore size of less than 2 nm and a porosity of less than 25%. The microporous sensitive layer 29 is at least partially made hydrophobic; in particular, the accessible micropores of the sensitive layer 29 are hydrophobic in nature.

The sensitive layer 29 is thus intended to be placed in the presence of the atmosphere to be tested, which may or may not be charged with ethanol, and more particularly the breath exhaled from an individual 14, that is to say the alveolar air saturated with water vapor, in order to allow a possible modification of the light intensity reflected by the reflective element 21 so as to determine the amount of ethanol contained in this atmosphere to be tested, in such a way as to obtain the blood alcohol content of the individual 14.

This is because, after the consumption of alcohol, the latter passes into the blood and spreads throughout the body and it is possible to detect its presence and to measure the blood alcohol content by measuring the ethanol contained in the alveolar air exhaled by an individual 14.

The light rays from the light source 23 are directed, in the present example, directly onto the sensitive layer 29 of the reflective element 21 in the presence of an atmosphere to be tested, which may or may not be charged with ethanol, and more particularly the breath exhaled from an individual 14. The rays are reflected toward a light detector 25 and, in the presence of ethanol, the refractive index of the reflective element 21 varies and makes it possible to deduce therefrom the blood alcohol content, by measuring the intensity of the reflected light as will be explained a little later.

The detection device 3 furthermore comprises a processing and calculation unit 31 connected to the light source 23 and to the light detector 25 for controlling the operation thereof and configured to deduce, from the intensity reflected by the sensitive layer 29, a parameter corresponding to a blood alcohol content. The processing and calculation unit 31 is, for example, also connected to a display unit 33 for displaying the blood alcohol content measured.

As the air inside the chamber 11 is replaced with the alveolar air from the individual 14, the ethanol possibly contained in the alveolar is adsorbed by the sensitive layer 29 whereas the adsorption of water is minimized because of the predominantly hydrophobic properties of the sensitive layer 29.

This reversible adsorption of ethanol brings about a reversible modification of the dielectric constant of the sensitive layer 29 and thus a change in the refractive index of the latter. Indeed, to simplify, the refractive index changes like the square root of the dielectric constant.

According to one particular embodiment of the invention, the inventors observed that, in the presence of a moisture-saturated atmosphere, the modifications of the reflectivity index of the reflective element were greater.

According to another embodiment of the invention, it would be advantageous to use the device in a medium for which the moisture content would be controlled.

According to one simplified approach with the Fresnel equations (see FIG. 2B) not taking into account the presence of the substrate 27, it is possible to put the following:

$$Rs = {\vert \frac{{n}_{1} cos ( {θ}_{i} ) - {n}_{2} cos ( {θ}_{t} )}{{n}_{1} cos ( {θ}_{i} ) + {n}_{2} cos ( {θ}_{t} )} \vert}^{2}$$

$$Rp = {\vert \frac{{n}_{1} cos ( {θ}_{t} ) - {n}_{2} cos ( {θ}_{i} )}{{n}_{1} cos ( {θ}_{t} ) + {n}_{2} cos ( {θ}_{i} )} \vert}^{2}$$

$$R = 1 \text{/} 2 ( Rs + Rp )$$

wherein
Rs represents the reflectivity for an incident polarization s,
Rp represents the reflectivity for an incident polarization p,
R represents the reflectivity in the case of nonpolarized light,
θᵢ represents the angle of the incident wave,
θₜ represents the angle of the transmitted wave,
θᵣ is the angle of detection of the light rays relative to the normal of the sensitive reflective element 21,
n₁ is the refractive index of air, n₁=1=constant,
n₂ is the refractive index of the sensitive layer 29 which varies as a function of the ethanol content contained in the atmosphere to be tested.

Given that, in this formula, only n₂ varies as a function of the ethanol content contained in the atmosphere to be tested, n₁, θ₁, θ₂ remaining constant, it is thus possible to deduce therefrom that the reflection rate decreases linearly with an increase in the refractive index n₂. It has been found that the refractive index n₂ varies linearly with the ethanol content adsorbed by the sensitive layer 29.

With the complex Fresnel equations taking into account the existence of the substrate 27 (which will be discussed later in a little more detail), a similar result is obtained, that is to say that the refractive index n₂ varies linearly with the ethanol content adsorbed by the sensitive layer 29.

The incident angles and the angles of detection θ₁, θ₂ are for example respectively between 30° and 75°. θ₁ and θ₂ may be equal (in the case of direct reflection), but are not necessarily so.

FIG. 3 is a graph representing a measurement showing a measurement voltage at the outlet of the light detector 25, which represents a parameter corresponding to the intensity reflected as a function of the ethanol concentration contained in the moist air.

The strong linear correlation between the two magnitudes is noted, such that, with a simple calibration, it is possible to quantify a volatile compound (in this case ethanol) in the atmosphere to be tested, in particular for measuring the blood alcohol content using the detection device 3.

Since, in the case of breath, the exhaled air is saturated with moisture (moisture content equal to 100%), it is not necessary to make a correction of the measurements as a function of the moisture content.

However, in certain cases of drift, a correction can be carried out using a second sensor sensitive to moisture, which will be explained later.

In addition, since the adsorption process is reversible, the detection device 3 is particularly suitable for repeated use, all the more so since the response time of the detection device 3 is less than a few seconds.

The properties of and the process for producing the sensitive layer 29 made of microporous hydrophobic sol-gel silica, will subsequently be detailed.

This layer has a thickness of greater than 250 nm, notably of between 400 nm and 1200 nm.

The mean pore size is less than 2 nm.

The mean pore diameter can be determined by means of a known method of volumetric analysis which is for example described in detail in the article “Porosity and mechanical properties of mesoporous thin films assessed by environmental ellipsometric porosimetry” Cedric Boissière et al, published in American Chemical Society, 2005. Langmuir: the ACS journal of surfaces and colloids 2005, 21, 12362-71.

The pore volume of the sensitive layer 29 placed on the substrate 27 is less than or equal to 25%. It is the volume fraction of the pore in the volume of the porous layer.

The surface area of accessible pores of the sensitive layer 29 placed on the substrate 27 is greater than 140 cm²/cm².

The surface area of accessible pores of the porous layer is determined in the following way:

Calculation of the total accessible pore surface area S in cm²/cm² (to be related back to one unit of surface area of s=1 cm²):

S=4*V*e*s/D

wherein
V is the pore volume, for example less than or equal to 25%,
e is the thickness of the hydrophobic porous layer 7, for example between 400 nm and 1000 nm, notably between 500 nm and 700 nm,
D is the mean pore size, for example less than or equal to 2 nm,
s unit surface area of 1 cm².

According to one embodiment, the sensitive layer 29 is produced without the addition of structuring agents, that is to say without the addition of chemical species of mineral or organic nature around which the material could organize. Examples of common structuring agents are notably CTAB (hexadecyltrimethylammonium bromide), DTAB (decyltrimethylammonium bromide) or F127 (the Pluronic F127 ([PEO]106-[PO]70-[PEO]106) triblock copolymer).

According to one preferred embodiment of the invention, the sensitive layer 29 is prepared by means of a sol-gel process for which the sol-gel sensitive layer 29 comprises at least one metal oxide made more hydrophobic, preferentially silica made more hydrophobic through the use of methyltriethoxysilane (MTEOS), ideally 50 mol %, and of tetraethyl orthosilicate (TEOS) used as silica precursor. Other routes of preparation by post-grafting or by using other agents that are methylated precursors of silica can be envisioned and are well known to those skilled in the art.

This sensitive layer 29 is deposited for example on just one face of the substrate 27.

According to one preferred embodiment, the solvent used for the preparation of the sensitive layer 29 is 2-propanol (PrOH); in particular, the solvent does not comprise ethanol (EtOH).

The sensitive layer 29 is then subjected to a stabilization treatment, the objectives of which are the consolidation of the inorganic network by condensation of the MTEOS and TEOS precursors to methylated SiO2 (or, if a metal oxide is used, to methylated oxo-metallic units) and the formation of the porosity by elimination of the volatile compounds, produced from the condensation, for example the water produced during the condensation of the MTEOS and TEOS precursors. This stabilization treatment is generally a heat treatment, for example a calcination at a temperature of between 200° C. and 400° C., notably 350° C. This results in pores of nanometric size.

This stabilization treatment can also be obtained by vapor-phase chemical treatment, for example by ammonia vapor, followed by leaching.

The sensitive layer 29 exhibits a variation in the refractive index as a function of the ethanol adsorbed. This variation may be about a few % in the range of measurement of the blood alcohol content, notably between 0-1% approximately.

The substrate 27 is chosen according to its capacity to withstand the conditions of the treatment and to have a surface chemistry which allows the formation of strong chemical bonds (of ionocovalent types) with the sensitive layer 29.

As already indicated above, the substrate 27 can be made of silicon.

This results in pores of very small size and a porosity of about 10-20% by volume, but a greater variation in the refractive index as a function of the ethanol adsorbed. This variation may be about a few % in the range of measurement of the blood alcohol content, notably between 0-1% approximately.

According to one example of the invention, microporous silica films have been prepared from a solution comprising tetraethoxysilane (TEOS)/MTEOS (methyltriethoxysilane)/HCl/H2O/PrOH with molar proportions of 0.5/0.5/0.17/4/5.8 respectively. The mean pore size is less than 2 nm.

The microporous films are obtained from 0.5 mol of TEOS and 0.5 mol of MTEOS which are first of all dissolved in a mixture composed of 5.8 mol of 2-propanol (PrOH), 0.17 mol of hydrochloric acid and 4 mol of water.

This solution can be stirred for, for example, at least 24 h at ambient temperature before being used. According to one variant, the solution is heated at 70° for approximately 30 minutes, then left to stand for a few hours, in particular between 3-5 h, at ambient temperature.

The solutions based on TEOS and on MTEOS can be combined with other types of precursors of metal oxides (organic metal, organometallic, salts) of other metal oxides, notably of the oxides of Ti, Al, Zr, Zn, Ca, Mg and/or Fe in any proportions.

Then, in order to obtain the sensitive layer 29, a sol-gel film of the solution previously described is deposited on the substrate 27 for example by liquid deposition (spin coating, by spraying, by ink jet printing, or dip coating) for example on just one face of the substrate 27.

The case of dip coating is preferred for the thickness uniformity.

The rate of withdrawal in the case of dip coating makes it possible to control the thickness of the sensitive layer 29 and is generally between 0.001 and 20 mm/s in order to adjust the thickness of the layer to the desired value.

The depositing of the sensitive layer 29 on the substrate 27 is carried out under a relative humidity, preferably between 10% and 99%, and even more preferentially of between 50% and 90% in order to obtain a layer having good ethanol adsorption properties.

The sensitive reflective element 25 comprising the substrate 27 and the sensitive layer 29 is then calcined at a temperature between 250° C. and 450° C., ideally at 350° C., for a period of time of greater than 5 min, notably for 10 min.

This heat treatment makes it possible to stabilize and consolidate the sensitive layer 29 by condensation of the TEOS and of the MTEOS to silica and to generate a porosity for which the mean size of the pores obtained is less than 2 nm.

In the case of the films obtained by co-condensation of TEOS and MTEOS and in order to ensure good hydrophobicity, a post-methylation can be carried out by immersing the reflective element 25, for 72 h, in a mixture of hexamethyldisilazane dissolved at 20 vol % in anhydrous toluene. The reflective element 25 is then again heat-treated at 350° C. for 10 min.

The device 3 for detecting a blood alcohol content operates by measuring the reflected intensity of the sensitive reflective element 25 as a function of the incident intensity emitted by the light source 23.

After calibration, it is possible to measure, with great accuracy, reliability and repeatability, the blood alcohol content of an individual 14 exhaling into the measuring chamber 11 of the detection device 3 and thus to generally quantify a volatile compound.

The detection device 3 also differs by virtue of the fact that it is not very bulky, and it has a rapid response time and a lower cost than the electronic breathalyzers known to date, while at the same time allowing great measurement reliability. It also makes it possible to detect small amounts of ethanol with accuracy, with a low threshold of approximately 0.02 mg/L of ethanol in moist air.

The sensitivity of the detection device 3 can also be optimized by a judicious choice of the thickness of the sensitive layer 29 and of the wavelength of the light source 23.

Indeed, simplified Fresnel equations were presented above to explain the basis of operation of the detection device 3.

In reality, these Fresnel equations integrate complex terms (i.e. integrate terms comprising the complex number i with i²=−1) and also take into account the substrate 27. These equations are solved digitally by computers.

FIG. 4 represents a graph showing, for two different thicknesses 200 nm (curve 100) and 700 nm (curve 102) of the sensitive layer 29, the reflectivity as a function of the wavelength A in nm.

These curves were obtained by digital solution of the complex Fresnel equations taking into account the presence of the substrate 27.

It is noted that the curve 100 corresponding to the thinner sensitive layer 29 exhibits, in the visible spectrum, just one constructive interference peak Ml, whereas the curve 102 corresponding to the thicker sensitive layer 29 exhibits three constructive interference peaks M′1, M′2 and M′3 and three destructive interference peaks D′1, D′2 and D′3. The position in terms of wavelength of the various peaks can vary as a function of the angle of incidence θi.

In the case of the reflection of a thin layer, the fact that the constructive and destructive interference peaks are narrower for thicker layers than for the thinner layers is a phenomenon known per se.

However, judiciously, it is possible to deduce, from these reflectivity spectra, one or more wavelengths which make it possible to have increased sensitivity of the detection device 3.

Indeed, FIG. 5 shows on a graph the variation in the reflected intensity as a function of the wavelength for a layer of a given thickness of 700 nm when the latter undergoes a variation in refractive index by a predefined value of 0.01.

It is noted that the maxima of different intensities allowing optimal analysis with great sensitive are given for the wavelengths λ₁, λ₂, λ₃ corresponding to one of the positions of the arrows of FIG. 4 which are located at the points of inflection between a constructive interference peak M′1, M′2 or M′3 and a destructive interference peak D′1, D′2 and D′3, these points of inflection being denoted for example I₁, I₂ and I₃, in the spectrum of FIG. 4.

In practice, the thickness e is chosen to be sufficiently large to enhance the slopes of the inflection zones of the reflection spectrum (as in FIG. 4), without the number of interferences exceeding a value that would make the alignment too difficult.

It proves to be judicious to thus choose a thickness e that makes it possible to have, in a wavelength range between 400 and 800 nm, at least two and at most three or four constructive interference peaks.

Moreover, too large a thickness e of the sensitive layer 29 would also bring about too long an equilibrium time that could be considered to be an impairment for a user of the detection device 3.

Since the wavelength of the light source 23 is monochromatic or quasi-monochromatic and fixed, the angle of incidence is then adjusted such that the position in terms of wavelength of an inflection of the spectrum coincides with the wavelength of the light source 23.

It is possible to determine these specific wavelengths for a sensitive layer with a given thickness e having a refractive index n₂ and deposited on a substrate 27 having a refractive index n₃ and also an angle of incidence θi by calculating the reflection spectrum R(λ) with the complex equations and by determining the points of inflection, for example I₁, I₂, or I₃ by determining the points for which the second derivative of the spectrum is equal to zero

$$( \frac{{\partial }^{2} R}{\partial {λ}^{2}} = 0 ) .$$

The points of inflection can also be determined experimentally by illuminating for example the reflective element 21 by means of a light source with a continuous and parallel spectrum, under a given angle of incidence, and by measuring the reflectivity as a function of the wavelength for example with a spectrometer, then by determining the points for which the second derivative of the spectrum measured is equal to zero

$$( \frac{{\partial }^{2} R}{\partial {λ}^{2}} = 0 ) .$$

The detection device 3 has a layer of substrate 27 having for example a refractive index ns, such that 1.8<n₃<4, notably 2.5<n₃<3.5, a sensitive layer 29 with a thickness of between 500 nm and 3000 nm, notably between 400 nm and 1000 nm, and having a refractive index n₂ such that 1.2<n₂<1.6, more particularly 1.3<n₂<1.4 for a wavelength λ of the light source 23 of between 500 nm and 1000 nm.

The method described above makes it possible to optimize the sensitivity of an optical detection device 3 based on the measurement of the reflectivity of a sensitive layer 3 deposited on a substrate 27.

The applicant declares that an independent protection can be sought for an optical detection device 3 based on a similar principle comprising a layer 29 sensitive to a specific compound and a substrate 27 which may be suitable for detection by reflectivity measurement, containing notably volatile compounds, in particular volatile organic compounds, notably aldehydes or aromatic compounds.

It is thus understood that the sensitivity of the detection device 3 can be enhanced by judiciously choosing in particular, on the one hand, the thickness of the sensitive layer and, on the other hand, the wavelength combined with the angle of incidence.

On a principle that is similar, to that of the detection of alcohol, it is possible to produce a device for the detection of chemical compounds other than ethanol. In this case, the sensitive layer 23 must be suitable first to be sensitive to and specific for the chemical compound other than ethanol, for example volatile organic compounds (VOCs), notably aldehydes and/or aromatic compounds.

As mentioned above notably in relation to FIG. 3, in the case of an exhaled breath containing ethanol, the moisture content is 100% and it was thus possible to observe a linear correlation between the measurement voltage at the outlet of the light detector 25 and the ethanol concentration contained in the moist air.

However, in certain more general cases, it may be that the moisture content differs from 100% such that a correction taking into account the influence of the moisture appears to be required in order to preserve a good accuracy of measurement. Furthermore, it has been noted that the outside temperature can also influence the measurement results and also drifts corresponding for example to the natural aging of the light source 23 or of the detector 25.

In this case, a new embodiment with another alternative processing of the measurement signals is proposed.

Represented in FIG. 6 is a measurement chamber 11 in which, similarly to FIG. 1, an individual 14 blows by exhaling their breath. Of course, more broadly, any flow of a volatile compound, notably volatile organic compound VOC, can be introduced into the measurement chamber 11, provided that the layer is sensitive to the volatile compound to be detected.

The light source 23A corresponds to the light source 23 of FIG. 1. The light detector 25A corresponds to the light detector 25 of FIG. 1 and the sensitive reflective element 21A corresponds to the sensitive reflector 21 of FIG. 1.

In this embodiment, the optical detection device 3 also comprises an additional sensitive reflective element 21B. This reflector 21B is produced in the same way and according to the same protocol as the reflector 21A with the only difference being that 100% of TEOS precursor and 0% of MTEOS precursor is used.

The additional sensitive reflective element 21B is illuminated an additional light source 23B and the reflected light is detected by the additional light detector 25B. The assembly between the additional light source 23B, the additional reflective element 23B and the additional light detector 25B is in all respects similar to the assembly of the light source 23A, the reflective element 23A and the light detector 25A and thus to the assembly as explained with regard to FIG. 1; only the angles of incidence and of detection can be different.

According to one variant, just one light source is used and the reflective elements are placed side by side so as to be illuminated by the same light source.

In this case, as discussed above, the sensitive layer 29 of the reflector 21A has a hydrophobic nature, while the sensitive layer of the reflector 21B, made from a 100% TEOS sol-gel solution, is hydrophilic. The result of this is that the reflector 21B does not (or virtually does not) absorb ethanol and that the variation in its refractive index is a function only of the moisture in the atmosphere to be tested. Thus, the measurement signal at the output of the light detector 25B varies as a function of the moisture content only.

FIG. 7 shows on a graph, as a function of time, a measurement signal 250A, for example a voltage, corresponding to the intensity of the light reflected by the reflective element 21A and a measurement signal 250B, for example a voltage, corresponding to the intensity of the light reflected by the reflective element 21A.

At an instant t1, for a period of 20 s up to the instant t2, water vapor was injected into the chamber 11. It is seen that the reflected intensity decreases abruptly for the two light detectors 25A and 25B. Then, after the injection has stopped, the measurement signals 250A and 250B increase, which corresponds to a re-equilibration with the surrounding atmosphere.

At an instant t₃, for a period of 20 s up to an instant t₄, a mixture of water vapor and 0.92 mg/L of ethanol was injected into the chamber 11. It is seen that the reflected intensity decreases abruptly for the two light detectors 25A and 25B, but in a more pronounced manner for the light detector 25A which measures the light reflected from the reflective element 21A. Then, after the injection has stopped, the measurement signals 250A and 250B increase, which corresponds to a re-equilibration with the surrounding atmosphere.

In order to obtain the ethanol content, it is necessary to process the measurement signals in the following way:

Let U_{25A}(t) be the change in the measurement signal over time of the light detector 25A (this thus corresponds to the curve 250A) and let U_{25B}(t) be the change in the measurement signal over time of the light detector 25B (this thus corresponds to the curve 250B).

In this case, the difference in the time derivative of the two intensities measured is determined:

$$f ( t ) = \frac{{dU}_{25 A}}{dt} - \frac{{dU}_{25 B}}{dt}$$

The result of this operation is shown in FIG. 8.

In the case of an absence of ethanol between t1 and t2, a plateau with a duration of Δt, which is also referred to as predetermined time zone of interest, for which the time derivative is the same for the two intensities measured, is observed.

$$Δ t = time interval with \frac{{dU}_{25 A}}{dt} - \frac{{dU}_{25 B}}{dt} = 0 in the absence of ethanol$$

In order to determine the ethanol content between t3 and t4, the mean of f(t) will be determined during the predetermined time zone of interest:

$$\overline{{f ( t )}_{Δ t}} = {\int }_{Δ t} f ( t ) dt$$

This mean ƒ̅(̅t̅)̅_̅{̅Δ̅t̅}̅ can be correlated by a correlation curve 252 shown in FIG. 9 in order to determine the ethanol content of the atmosphere to be tested. This calibration curve 252 can be determined experimentally, and can then be stored or recorded in digital form in the processing and calculation unit 31.

This embodiment can be considered to be more robust, since it allows corrections not only to take into account the influence of the moisture, but also to take into account the drifts associated with the outside temperature, with the conditions for placing in the presence of the exhaled air and with the natural aging of the various components forming the optical detection device 3.

1. An optical device for detecting and quantifying volatile compounds, comprising:
a sensitive reflective element, the reflection rate of which varies as a function of the ethanol content contained in an atmosphere to be tested, the sensitive reflective element comprising:
a substrate,
a sensitive layer comprising microporous hydrophobic sol-gel silica having a thickness of greater than 250 nm, notably of between 400 nm and 1200 nm, a mean pore size of less than 2 nm and a porosity of less than 25%, the sensitive layer being intended to be placed in the presence of an atmosphere to be tested, notably which may or may not be charged with ethanol, and more particularly the breath exhaled from an individual,
a monochromatic or quasi-monochromatic light source arranged to illuminate the sensitive layer under an incident angle,
a light detector for measuring the intensity reflected by the reflective element under an angle of detection,
and
a processing and calculation unit configured to deduce from the intensity reflected by the reflective element a parameter corresponding to a blood alcohol content.

2. The detection device as claimed in claim 1, wherein the sensitive layer does not comprise structuring agents, notably CTAB, DTAB or F127.

3. The detection device as claimed in claim 1, wherein the substrate has a refractive index greater than 2.5, notably greater than 3 for a wavelength of between 250 nm and 1500 nm.

4. The detection device as claimed in claim 1, wherein the substrate is made of a semiconductive material, notably of silicon.

5. The detection device as claimed in claim 1, wherein the incident angle and the angle of detection are respectively between 30° and 75°.

6. The detection device as claimed in claim 1, wherein the wavelength of the light source is monochromatic and chosen with the angle of incidence so as to coincide with the position in terms of wavelength of an inflection between two constructive and destructive interference peaks of the reflection spectrum of the reflective element.

7. The detection device as claimed in claim 6, wherein the wavelength of the light source is between 500 nm and 1000 nm.

8. The detection device as claimed in claim 1, wherein the sensitive layer has a refractive index of between 1.2 and 1.6, more particularly between 1.3 and 1.4 for a wavelength between 500 nm and 1000 nm.

9. The detection device as claimed in claim 1, wherein it also comprises
an additional sensitive reflective element, the reflection rate of which varies as a function of the moisture content contained in an atmosphere to be tested,
an additional light detector for measuring the intensity reflected by the additional sensitive reflective element under an angle of detection,
the processing and calculation unit being configured to deduce from the intensities reflected, on the one hand, by the reflective element and, on the other hand, by the additional sensitive reflective element a parameter corresponding to a blood alcohol content while taking into account the influence of the moisture in the atmosphere to be tested.

10. A process for producing a sensitive reflective element for an optical device for detecting and quantifying volatile compounds as claimed in claim 1, wherein
a sol-gel solution is prepared by dissolving TEOS and MTEOS in a solution composed of PrOH, hydrochloric acid and water,
the sol-gel solution is deposited on the substrate under a relative humidity of between 40% and 90%, in particular between 50% and 60% in order to obtain a sensitive layer,
the sensitive layer is subjected to a calcining step at a temperature of between 250° C. and 450° C., notably at 350° C., this being for a period of time of greater than 5 min, notably for 10 min.

11. The process for producing a sensitive reflective element as claimed in claim 10, wherein the thickness of the sensitive layer is greater than 250 nm, notably between 400 nm and 1200 nm.

12. A sensitive reflective element for an optical device for detecting and quantifying volatile compounds as claimed in claim 1, wherein the reflective element comprises
a substrate, and
a sensitive layer comprising microporous hydrophobic sol-gel silica having a thickness of greater than 250 nm, notably of between 400 nm and 1200 nm, a mean pore size of less than 2 nm and a porosity of less than 25%, the sensitive layer being intended to be placed in the presence of an atmosphere to be tested, notably which may or may not be charged with ethanol, and more particularly the breath exhaled from an individual.

13. The sensitive reflective element as claimed in claim 12, wherein a reflection rate which varies as a function of the ethanol content contained in an atmosphere to be tested.

14. A process for producing an optical detection device for detecting and quantifying volatile compounds as claimed in claim 1, wherein
a sensitive layer is deposited on a substrate in order to form a sensitive reflective element,
the reflection spectrum of the sensitive reflective element is determined,
the wavelength of the light source is monochromatic, or quasi-monochromatic, and chosen with the angle of incidence of the light ray on the reflective element) so as to coincide with the position in terms of wavelength of an inflection (I₁, I₂, I₃) of the reflection spectrum of the reflective element.

15. The production process as claimed in claim 14, wherein the substrate has a refractive index of between 1.8 and 4, notably between 2.5 and 3.5, the sensitive layer has a thickness of greater than 250 nm, notably of between 400 nm and 1200 nm, and has a refractive index of between 1.2 and 1.6, more particularly between 1.3 and 1.4 and the wavelength of the light source is between 500 nm and 1000 nm.

16. The process as claimed in claim 14, wherein the reflective element is produced according to the process for producing a sensitive reflective element for an optical device for detecting and quantifying volatile compounds, said optical device comprising a sensitive reflective element, the reflection rate of which varies as a function of the ethanol content contained in an atmosphere to be tested, the sensitive reflective element comprising:
a substrate,
a sensitive layer comprising microporous hydrophobic sol-gel silica having a thickness of greater than 250 nm, notably of between 400 nm and 1200 nm, a mean pore size of less than 2 nm and a porosity of less than 25%, the sensitive layer being intended to be placed in the presence of an atmosphere to be tested, notably which may or may not be charged with ethanol, and more particularly the breath exhaled from an individual,
a monochromatic or quasi-monochromatic light source arranged to illuminate the sensitive layer under an incident angle,
a light detector for measuring the intensity reflected by the reflective element under an angle of detection,
and
a processing and calculation unit configured to deduce from the intensity reflected by the reflective element a parameter corresponding to a blood alcohol content, wherein
a sol-gel solution is prepared by dissolving TEOS and MTEOS in a solution composed of PrOH, hydrochloric acid and water,
the sol-gel solution is deposited on the substrate under a relative humidity of between 40% and 90%, in particular between 50% and 60% in order to obtain a sensitive layer,
the sensitive layer is subjected to a calcining step at a temperature of between 250° C. and 450° C., notably at 350° C., this being for a period of time of greater than 5 min, notably for 10 min.

17. An optical detection process for detecting and quantifying volatile compounds in an atmosphere to be tested, the optical detection process comprising:
measuring the intensity reflected by a sensitive reflective element comprising:
a substrate,
a sensitive layer comprising microporous hydrophobic sol-gel silica having a thickness of greater than 250 nm, notably of between 400 nm and 1200 nm, a mean pore size of less than 2 nm and a porosity of less than 25%, the sensitive layer being intended to be placed in the presence of an atmosphere to be tested, notably which may or may not be charged with ethanol, and more particularly the breath exhaled from an individual,
a monochromatic or quasi-monochromatic light source arranged to illuminate the sensitive layer under an incident angle,
a light detector for measuring the intensity reflected by the reflective element under an angle of detection, and
a processing and calculation unit configured to deduce from the intensity reflected by the reflective element a parameter corresponding to a blood alcohol content
the intensity reflected by an additional sensitive reflective element as claimed in claim 9, the reflection rate of which varies as a function of the moisture content contained in the atmosphere to be tested, is measured,
in a predetermined time zone of interest (Δt) for which the time derivative is the same for the two intensities measured in the case of the absence of ethanol, the difference in the time derivative of the two intensities measured is determined in order to carry out a correlation with a calibration curve in order to quantify the ethanol contained in the atmosphere to be tested.

18. The optical detection process for detecting and quantifying volatile compounds in an atmosphere to be tested as claimed in claim 17, wherein the wavelength of the light source is chosen so as to coincide with the position in terms of wavelength of an inflection between two constructive and destructive interference peaks of the reflection spectrum of the sensitive reflective element.
